(19) United States
(12) Patent Application Publication
(10) Pub. No.: US 2018/0057782 A1
(43) Pub. Date: Mar. 1, 2018

(54) LOW ENERGY PHOTOBIOREACTOR SYSTEM

(71) Applicant: Syed Saad Ahsan, Hillsboro, OR (US)

(72) Inventor: Syed Saad Ahsan, Hillsboro, OR (US)

(21) Appl. No.: 15/436,713

(22) Filed: Feb. 17, 2017

Related U.S. Application Data
(60) Provisional application No. 62/380,318, filed on Aug. 26, 2016.

(51) Int. Cl. C12M 1/00

(52) U.S. Cl. CPC C12M 21/02; C12M 23/22

(57) ABSTRACT

The present disclosure relates to a photobioreactor system. Algae for using producing alternative energy is typically grown using circular raceway ponds, tubular photobioreactors, and/or ultracompact waveguide photobioreactors. These methods require a supply of conventional energy for mixing, pumping fluids, distributing light, and/or other operations. The conventional energy required for these algae growth methods is high compared to the alternative energy eventually harvested from a particular system. Thus, the energy return on investment for these systems is low. The present photobioreactor system facilitates passive fluid flow through growth chambers. The passive fluid flow may be caused by gravity. Advantageously, the present system may decrease the energy costs of cultivating algae and/or other biomasses using photobioreactors because the present system requires little to no power for operation. In some embodiments, the present system may comprise growth chambers, an influent device, an effluent device, a support structure, and/or other components.

### CROSS-REFERENCE TO RELATED APPLICATIONS

This application claims the benefit of U.S. Provisional Patent Application No. 62/380,318, filed Aug. 26, 2016, entitled “LOW ENERGY PHOTOBIOREACTOR SYSTEM,” the contents of which is incorporated herein by reference in its entirety.

### BACKGROUND OF THE INVENTION

### 1. Field of the Invention

The present disclosure relates to a photobioreactor system configured to grow a biomass. The photobioreactor system is configured such that influent fluid passively flows from an influent device into a growth chamber, and effluent fluid, including a portion of the biomass, passively flows from the growth chamber into an effluent device.

### 2. Description of Related Art

Growing algae for use as a source of alternative energy is known. Algae is typically grown using circular raceway ponds, tubular photobioreactors, and/or ultracompact waveguide photobioreactors. These methods require a supply of conventional energy for mixing, pumping fluids, distributing light, and/or other operations. The conventional energy required for these algae growth methods is relatively high compared to the alternative energy eventually harvested from a particular system. Thus, the energy return on investment (EROI) for prior art systems is low.

### SUMMARY OF EMBODIMENTS OF THE INVENTION

One aspect of the present disclosure relates to a photobioreactor system. The photobioreactor system comprises a growth chamber, an influent device, an effluent device, a support structure, and/or other components. The growth chamber may be configured to grow a biomass. The growth chamber may comprise a layered structure and/or other structures. The layered structure may include an adsorbent material layer coupled to a first semi-permeable surface on a first side of the adsorbent material layer and a second semi-permeable surface on a second side of the adsorbent material layer. The second side may be opposite the first side. The first and second semi-permeable surfaces may facilitate biomass growth in the adsorbent material layer by permitting passive input and output of fluid and gases to and from the growth chamber and/or other operations. The influent device may be configured to provide influent fluid to the growth chamber. The effluent device may be configured to receive effluent fluid from the growth chamber. The support structure may be configured to support the growth chamber, the influent device, and the effluent device such that: the influent fluid passively flows from the influent device into the growth chamber and the effluent fluid passively flows from the growth chamber into the effluent device; gases passively move through the first and second semi-permeable surfaces into and out of the growth chamber; and visible light passes through the first and second semi-permeable surfaces into the growth chamber.

A second aspect of the present disclosure relates to a method for growing a biomass with a photobioreactor system. The photobioreactor system may comprise a growth chamber, an influent device, an effluent device, a support structure, and/or other components. The method may comprise: forming, with the growth chamber, a layered structure, the layered structure including an adsorbent material layer coupled to a first semi-permeable surface on a first side of the adsorbent material layer and a second semi-permeable surface on a second side of the adsorbent material layer, the second side opposite the first side, facilitating, with the first and second semi-permeable surfaces, biomass growth in the adsorbent material layer by permitting passive input and output of fluid and gases to and from the growth chamber; providing, with the influent device, influent fluid to the growth chamber; receiving, with the effluent device, effluent fluid from the growth chamber; and supporting, with the support structure, the growth chamber, the influent device, and the effluent device such that: the influent fluid passively flows from the influent device into the growth chamber and the effluent fluid passively flows from the growth chamber into the effluent device; gases passively move through the first and second semi-permeable surfaces into and out of the growth chamber; and visible light passes through the first and second semi-permeable surfaces into the growth chamber.

In some embodiments, the support structure may be configured such that the passive influent fluid flow from the influent device into the growth chamber and the passive effluent fluid flow from the growth chamber into the effluent device are caused by gravity. In some embodiments, the support structure may be configured such that the growth chamber is oriented vertically, the influent device is positioned above the growth chamber, and the effluent device is positioned below the growth chamber. In some embodiments, the biomass may be algae. In some embodiments, the first and second semi-permeable surfaces may permit passive input and output of carbon dioxide, oxygen, water vapor, the influent fluid, and the effluent fluid into and/or out of the growth chamber to facilitate biomass growth. In some embodiments, the first and second semi-permeable surfaces may be transparent or translucent to visible light. In some embodiments, the adsorbent material layer may become translucent to visible light responsive to being wetted by the influent fluid. In some embodiments, the adsorbent material layer may comprise a fibrous material including one or more of tissue paper, cotton, or a sponge. In some embodiments, the adsorbent material layer may be between about 30 μm and about 1 cm thick. In some embodiments, the first and second semi-permeable surfaces may comprise polydimethylsiloxane (PDMS) and/or other materials. In some embodiments, the first and second semi-permeable surfaces may be between about 20 μm and about 1 cm thick. In some embodiments, the influent device may provide the influent fluid to the growth chamber via a first needle that couples the influent device to the growth chamber, and the effluent device may receive the effluent fluid from the growth chamber via a second needle that couples the growth chamber to the effluent device. In some embodiments, the influent device may be configured to provide a continuous flow of influent fluid. In some embodiments, the influent device comprises a flow controller configured to control a flow rate of the influent fluid to the growth chamber. In some embodiments, the effluent device is configured to collect the biomass from the effluent fluid.

These and other aspects of various embodiments of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein are drawn to scale. It is to be expressly understood, however, that the drawings are for the purpose of illustration and description only and are not intended as a definition of the limits of the invention. In addition, it should be appreciated that structural features shown or described in any one embodiment herein can be used in other embodiments as well. As used in the specification and in the claims, the singular form of “a”, “an”, and “the” include plural referents unless the context clearly dictates otherwise.

All closed-ended (e.g., between A and B) and open-ended (greater than C) ranges of values disclosed herein explicitly include all ranges that fall within or nest within such ranges. For example, a disclosed range of 1-10 is understood as also disclosing, among other ranged, 2-10, 1-9, 3-9, etc.

### BRIEF DESCRIPTION OF THE DRAWINGS

For a better understanding of embodiments of the present invention as well as other objects and further features thereof, reference is made to the following description which is to be used in conjunction with the accompanying drawings, where:

FIG. 1 illustrates a photobioreactor system;

FIG. 2 illustrates a layered structure of a growth chamber of the photobioreactor system; and

FIG. 3 illustrates a method for growing a biomass with a photobioreactor system.

### DETAILED DESCRIPTION OF EXEMPLARY EMBODIMENTS OF THE INVENTION

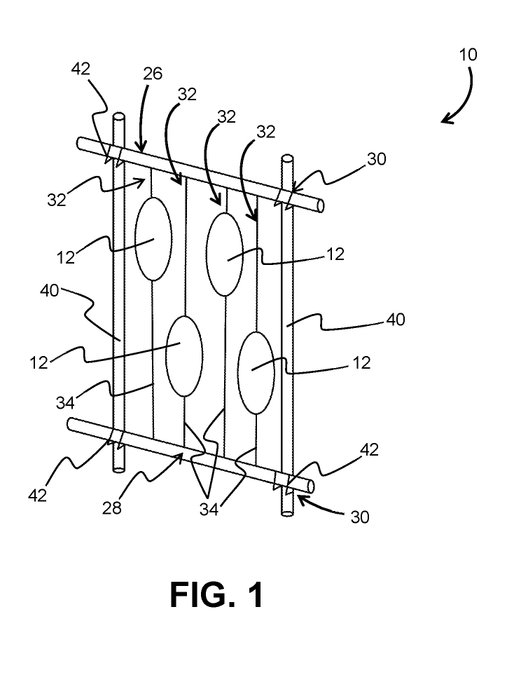

FIG. 1 illustrates a photobioreactor system 10. Algae and/or other biomasses may be used as a source of alternative energy. System 10 may be configured to facilitate growth of algae and/or other biomasses. As described above, algae is typically grown using circular raceway ponds, tubular photobioreactors, and/or ultracompact waveguide photobioreactors which require a supply of conventional energy for mixing, pumping fluids, distributing light, and/or other operations that lowers the EROI for these systems.

Circular raceway ponds and tubular reactors may be unable to efficiently distribute nutrients, such as carbon dioxide, and remove waste, such as oxygen, uniformly due to poor mixing and/or other factors. These systems may also be unable to distribute light in the bioreactor uniformly due to self-shading (as one example) so that only a thin (middle) layer of the pond receives the optimal light intensity. A top layer may receive too much light which destroys their photo-synthetic machinery by creating radical species, and a bottom layer may not get enough light to sustain growth. These systems require energy-intensive mixing to cycle algae to optimal light zones from the non-optimal zones and to better distribute carbon dioxide and remove oxygen. Even in simple closed photobioreactor systems, the energy required for mixing is close to the energy that is eventually produced by the system.

Ultracompact waveguide photobioreactors deliver light through waveguides. In these systems light is released uniformly into a photobioreactor using embedded scatterers. However, these systems are unable to yield enough algae to make them competitive with systems like the pond reactors describe above. Overall, a shared disadvantage of prior art systems is the power required to run the system (e.g., to pump media around the system to distribute carbon dioxide and remove oxygen, to power a light source, etc.).

In contrast to prior art systems, system 10 facilitates passive fluid flow through growth chambers. Advantageously, system 10 may decrease the energy costs of cultivating algae and/or other biomasses using photobioreactors because system 10 requires little to no power for operation. System 10 may be configured to continuously generate algal and/or other biomasses collected from effluent liquid. System 10 may allow for cheaper generation of the algal biomass which may, in turn, be used to create other useful gaseous and/or liquid products.

System 10 is described herein with respect to algae growth. However, system 10 may be used to facilitate any photo reaction, chemical and/or catalytic, where: 1) the optimal reaction condition is sensitive to light intensity such that normal sunlight is too intense, and 2) the reaction requires high mass transfer of reactants and/or products that are gaseous. For example, system 10 may be used as and/or with a photocatalytic device producing a gaseous output (e.g., hydrogen) where the catalysis is saturated and/or damaged at higher light intensities and/or reaction rates are reduced at higher concentrations of the gaseous product in the solution. In such a case, the catalyst may be embedded so that it would not be collected but that the gaseous product which escapes the reactor passively is collected.

In some embodiments, system 10 may comprise one or more of a growth chamber 12, an influent device 26, an effluent device 28, a support structure 30, and/or other components.

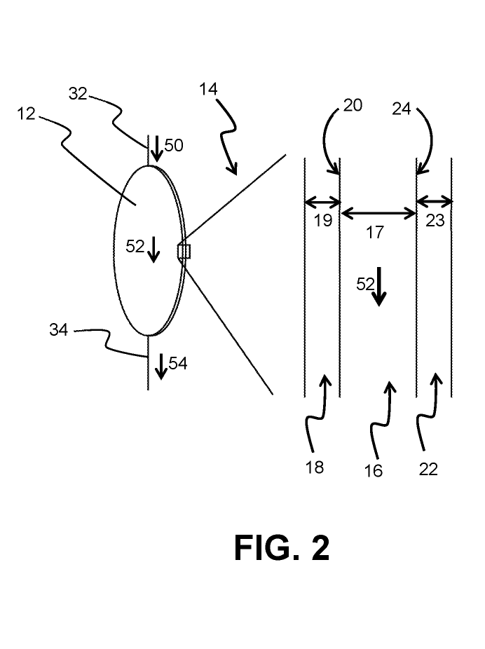

As shown in FIG. 1, system 10 may include a plurality of growth chambers 12. Four growth chambers 12 are shown as an example in FIG. 1, but this is not intended to be limiting. System 10 may include any number of growth chambers 12 that allows system 10 to function as described herein. Growth chambers 12 may be configured to grow a biomass. The biomass may be photosynthetic cells, algae, and/or other substances (e.g., as described above). In some embodiments, the photosynthetic cells and/or algae may be a wild species and/or genetically modified. In some embodiments, the photosynthetic cells and/or algae may secrete biofuels and/or other substances. An individual growth chamber 12 may comprise a layered structure and/or other structures. The layered structure 14 is illustrated in FIG. 2.

As shown in FIG. 2, layered structure 14 may including an adsorbent material layer 16 coupled to a first semi-permeable surface 18 on a first side 20 of adsorbent material layer 16, and a second semi-permeable surface 22 on a second side 24 of adsorbent material layer 16. The second side 24 of adsorbent material layer 16 is opposite first side 20. Layered structure 14 may be between about 70 μm and about 3 cm thick and/or have other thicknesses. First and second semi-permeable surfaces 18 and 22 may facilitate biomass growth in adsorbent material layer 16 by permitting passive input and output of fluid and gases to and from an individual growth chamber 12. For example, first and second semi-permeable surfaces 18 and 22 may permit passive input and output of carbon dioxide, oxygen, water vapor, influent fluid, effluent fluid, and/or other liquids and/or gasses into and/or out of a growth chamber 12 to facilitate biomass growth. In addition, first and second semi-permeable surfaces 18 and 22 may be transparent or translucent to visible light to facilitate growth. In some embodiments, first and second semi-permeable surfaces 18 and 22 may comprise polydimethylsiloxane (PDMS) and/or other materials. In some embodiments, first and second semi-permeable surfaces 18 and 22 may have thicknesses 19 and 23 between about 20 μm and about 1 cm, and/or have other thicknesses. In some embodiments, adsorbent material layer 16 may comprise a fibrous material including one or more of tissue paper, cotton, a sponge, and/or other materials. In some embodiments, adsorbent material layer 16 may become translucent to visible light responsive to being wetted by an influent fluid and/or other fluids. In some embodiments, adsorbent material layer 16 may have a thickness 17 between about 30 μm and about 1 cm, and/or may have other thicknesses.

By way of a non-limiting example, growth chambers 12 may be formed by applying a thin contiguous layer of PDMS on a substantially planar surface (e.g., a surface of a petri dish), with a volume that produces an average thickness of PDMS of about 30-40 μm. The PDMS layer may be allowed to settle and/or harden. The PDMS layer may then be coupled to tissue paper. In some embodiments, two PDMS layers may be sandwiched around the tissue paper and sewn together using string and/or other materials. Holes around the string (e.g., caused by the string passing through the PDMS layers) may be sealed using additional PDMS and/or other materials. In some embodiments, two PDMS layers may be sandwiched around the tissue paper and sewn together using meltable string and/or other materials. The meltable string may then be heated to melt to seal the holes created by the sewing. In some embodiments, a thinner layer of wet PDMS may be applied onto the dried and hardened PDMS layer while the layer is still attached to the substantially planar surface and/or at other times. The tissue paper may be applied to the wet surface and heated to bake the wet PDMS layer to form a connective layer between the dried PDMS layer and the tissue paper. This assembly may then be removed (e.g., peeled) from the substantially planar surface, and the process may be repeated on the other side of the tissue paper.

Returning to FIG. 1, influent device 26 may be configured to provide influent fluid to growth chambers 12. Influent device 26 may provide a nutrient supply to growth chambers 12 via the influent fluid. For example, influent device 26 may supply nitrogen, phosphorous, and/or other nutrients to growth chambers 12 via the influent fluid. In some embodiments, influent device 26 may be configured to supply carbonated media to growth chambers 12 via the influent fluid. In some embodiments, carbon dioxide may be provided to growth chambers 12 via environmental gasses surrounding system 10 (e.g., the carbon dioxide passes through firs and/or second semi-permeable surfaces 18 and 22. By way of a non-limiting example, the influent fluid may be and/or include a BG-11 medium (5×), 4.6 g/L (for example) TES buffer, and/or other materials. The carbon source may be provided by including an optional 20 mM NaHCO3 (for example) as an additional carbon source in the influent fluent, by enclosing growth chambers 12 and/or other components of system 10 in a sealed and/or semi-sealed environment (e.g., a greenhouse and/or other environments) and providing 1% CO2 in the air, or by providing other carbon sources.

In some embodiments, influent device 26 may comprise a channel, a tube, pipes, and/or other components. In some embodiments, influent device 26 may provide the influent fluid to an individual growth chamber 12 via a needle 32 and/or other conduits that couple influent device 26 to the individual growth chamber 12. In some embodiments, needles 32 and/or other conduits may be coupled with the channel(s), tube(s), pipe(s), and/or other components that form influent device 26 by way of one or more orifices in the channel(s), tube(s), pipe(s), and/or other components of influent device 26, and/or coupled by way of other features. For example, the one or more orifices may have an inner diameter slightly larger than an outer diameter of needles 32 such that an end of a needle may be inserted into an individual orifice. Such couplings may be sealed with PDMS and/or other materials, for example.

In some embodiments, influent device 26 may be configured to provide a continuous flow of influent fluid to growth chambers 12. In some embodiments, influent device 26 may be configured to provide a continuous drip flow of influent fluid to growth chambers 12. In some embodiments, the drip flow may be caused by hydrostatic pressure created and/or set by an arrangement of the channels, tubes, pipes, and/or other components of influent device 26 and/or effluent device 28, and/or created in other ways. In some embodiments, influent device 26 may comprise one or more flow controllers configured to control a flow rate of the influent fluid to growth chambers 12. The one or more flow controllers may comprise valves and/or other flow controllers.

Effluent device 28 may be configured to receive effluent fluid from growth chambers 12. In some embodiments, effluent device may comprise a channel, a tube, pipes, vials, and/or other components. Effluent device 28 may receive the effluent fluid from an individual growth chamber 12 via a needle 34 and/or other conduit that couples the individual growth chamber 12 to effluent device 28. In some embodiments, needles 32 and 34 may be inserted into the growth chambers 12 on opposite sides (e.g., as shown in FIG. 1) and/or in other locations to provide the influent fluid to growth chambers 12 and receive the effluent fluid from growth chambers 12.

By way of a non-limiting example, needles 32 and/or 34 may be and/or include a 23 gauge needle that has been blunted at its ends by torsion and/or other methods. The description of a 23 gage needle and/or the blunting by torsion is not intended to be limiting. Needles 32 and/or 34 may be formed by any conduit of any size that allows system 10 to function as described herein. Continuing with this example, needles 32 and/or 34 may be inserted into the opposite and/or other sides of a growth chamber 12 and sealed with PDMS and/or other materials. Needles 32 and/or 34 may be inserted into growth chambers 12 so as not to puncture the semi-permeable walls (first and second semi-permeable surfaces 18 and 22) of the photobioreactor.

In some embodiments, effluent device 28 may be configured to collect the biomass from the effluent fluid. By way of a non-limiting example, effluent device 28 may include (e.g., glass) vials for collecting the algae and/or other biomasses in the effluent fluid. The algae and/or other biomass may be collected with the effluent fluid that drips out of growth chambers 12 through needles 34 and/or other conduits, for example. As described above, the drip flow may be caused by hydrostatic pressure created and/or set by an arrangement of the channels, tubes, pipes, vials, and/or other components of influent device 26 and/or effluent device 28, and/or created in other ways.

Support structure 30 may be configured to support growth chambers 12, influent device 26, effluent device 28, and/or other components of system 10. In some embodiments, support structure 30 may be and/or include a frame, a net, a cage, shelves, a wall, boxes, benches, and/or other support structures. Support structure 30 may be configured to support growth chambers 12, influent device 26, effluent device 28, and/or other components of system 10 such that the influent fluid passively flows from influent device 26 into growth chambers 12 and the effluent fluid passively flows from growth chamber 12 into effluent device 28. Support structure 30 may be configured to support growth chambers 12, influent device 26, effluent device 28, and/or other components of system 10 such that liquids and/or gases passively move through first and second semi-permeable surfaces 18 and 22 (shown in FIG. 2) into and out of growth chambers 12. Support structure 30 may be configured to support growth chambers 12, influent device 26, effluent device 28, and/or other components of system 10 such that visible light passes through first and second semi-permeable surfaces 18 and 22 (FIG. 2) into growth chambers 12. In some embodiments, support structure 30 may be configured such that that growth chambers are oriented east to west (for example) and/or have other arrangements that correspond to a daily path of the sun. In such embodiments, light may be scattered off the ground and/or other surfaces by a diffusively reflective material (e.g., white paint), that may distribute the light so that the flux light intensity going through individual growth chambers 12 may be reduced. Advantageously, such embodiments facilitate operation of system 10 at a more ideal regime of light intensity for the algae, and the cost of redistributing light in this way is significantly smaller than methods used by prior art systems (e.g., in waveguide reactors). In some embodiments, support structure 30 may be configured such that the passive influent fluid flow from influent device 26 into growth chambers 12 and the passive effluent fluid flow from growth chambers 12 into effluent device 28 are caused by gravity. In some embodiments, support structure 30 may be configured such that growth chambers 12 are positioned sided by side in a two dimensional array and/or in other configurations.

For example, as illustrated in FIG. 1 and in FIG. 2, support structure 30 may be configured such that growth chambers 12 are oriented vertically. Support structure 30 may be configured such that gravity causes passive flow 50 (FIG. 2) of influent fluid into growth chamber 12, through 52 (FIG. 2) growth chamber 12, and out 54 (FIG. 2) of growth chamber 12 as effluent fluid. In some embodiments, influent device 26 (FIG. 1) is positioned above growth chambers 12, and effluent device 28 is positioned below growth chambers 12. In FIG. 1, influent device 26 is oriented substantially horizontally above growth chambers 12 and effluent device 28 is oriented substantially horizontally below growth chambers 12. In some embodiments, influent device 26 may be positioned substantially level with a growth chamber 12 and effluent device 28 may be positioned below the growth chamber 12. In some embodiments, influent device 26 may be placed above a growth chamber 12 and effluent device 28 may be positioned substantially level with the growth chamber 12. In some embodiments, influent device 26 and effluent device 28 are coupled via support members 40. In some embodiments, support structure 30 may include fittings, joints, hinges, screws, nuts, bolts, nails, adhesive, hooks, clamps, and/or other components 42 for coupling and/or otherwise supporting influent device 26, effluent device 28, support members 40, needles 32 and 34, growth chambers 12, and/or other components of system 10 as described herein. It should be noted that the number of support members 40, the components 42 described above and the corresponding illustration in FIG. 1 is not intended to be limiting. System 10 and/or support structure 30 may include any number and/or type of support members 40 and/or components 42 that allow system 10 to function as described herein. For example, support structure 30 may not include support members 40 at all in embodiments where influent device 26 and effluent device 28 are coupled with a wall and/or some other support structure 30.

In some embodiments, system 10 includes sensors and/or other devices for monitoring biomass growth in system 10. For example, in some embodiments, system 10 may include temperature sensors, pH sensors, humidity sensors, light level sensors, oxygen sensors carbon dioxide sensors, and/or other devices. In some embodiments, system 10 includes one or more devices configured to control system 10 and/or the environment surrounding system 10 based on information in output signals from such sensors. For example, system 10 may include one or more hardware processors configured by machine readable instructions to control a light level of lights in a room (and/or other location) that houses system and/or an amount of light output by a lighting device near system 10, control a heating/cooling device to adjust the temperature of the room, control a humidifier to adjust a humidity of the environment around system 10, control the valve in influent device 26 to adjust the flow rate of the influent fluid flowing to growth chambers 12, control a composition of gases (e.g., control a percentage of oxygen and/or a percentage of carbon dioxide) in the environment around system 10, and/or control other parameters. In some embodiments, the room may be sealed from an outside environment to decrease a loss of carbon dioxide during operation of system 10 and/or for other reasons.

By way of a non-limiting example, system 10 may be used to grow a Botorycoccus Braunii species and/or other species of algae. In some embodiments, the algae may be initially grown in flasks using a carbonated culture media similar to and/or the same as the influent fluid described herein. The algal species may then be inserted into growth chambers 12 and allowed to settle into interstitial spaces in the adsorbent material (e.g., tissue paper, cloth, cotton, polyester wipes, and/or any other inexpensive adsorbent material capable of transmitting fluid and supporting algae growth) for about 24 hours (for example). After this period of time, growth chambers 12 may be suspended vertically and coupled with influent device 26 and effluent device 28 to facilitate influent fluid to flow through growth chambers 12 caused by gravity. In this example, light may be provided by an incandescent lamp. Botorycoccus Braunii and/or other algae may grow slowly (e.g., having a doubling time of about 2-3 weeks). In this example, growth chambers 12, which were initially seeded with the dilute suspension of the algae, may facilitate growth of the algae as described herein for a duration of a month or more. Algae growth during the month or more period of time may be indicated by the color of the effluent collected daily by system 10 and/or other factors. In general, the light intensity of the light from the incandescent lamp for algal growth may be about a fifth to a tenth of what is available from the sun. The adsorbent material of layer 16 that becomes translucent when wet, the thickness of semi-permeable surfaces 18 and 22, and/or other features of system 10 facilitate diffusive light distribution throughout growth chambers 12, carbon dioxide diffusion into growth chambers 12, diffusion of the oxygen out of growth chambers 12, and/or other operations.

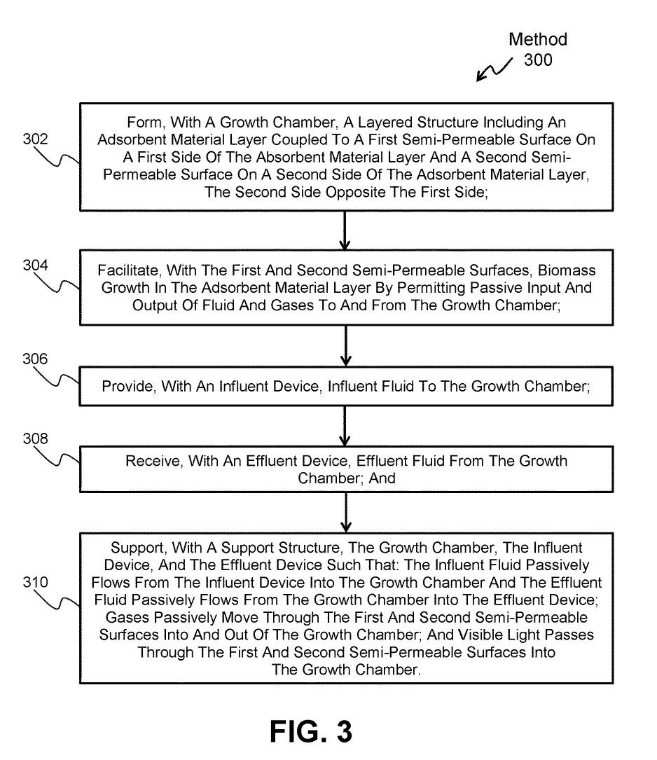

FIG. 3 illustrates a method 300 for growing a biomass with a photobioreactor system. In some embodiments, the biomass may be algae. The photobioreactor system may comprise a growth chamber, an influent device, an effluent device, a support structure, and/or other components. The operations of method 300 presented below are intended to be illustrative. In some embodiments, method 300 may be accomplished with one or more additional operations not described, and/or without one or more of the operations discussed. Additionally, the order in which the operations of method 300 are illustrated in FIG. 3 and described below is not intended to be limiting.

At an operation 302, a layered structure may be formed with the growth chamber. The layered structure may include an adsorbent material layer coupled to a first semi-permeable surface on a first side of the adsorbent material layer and a second semi-permeable surface on a second side of the adsorbent material layer. The second side may be opposite the first side. In some embodiments, operation 302 may be performed by a growth chamber that is the same as or similar to growth chamber 12 (shown in FIG. 1 and described herein).

At an operation 304, biomass growth in the adsorbent material layer may be facilitated with the first and second semi-permeable surfaces. Biomass growth may be facilitated by permitting passive input and output of fluid and gases through the first and second semi-permeable surfaces to and from the growth chamber. In some embodiments, the first and second semi-permeable surfaces may permit passive input and output of carbon dioxide, oxygen, water vapor, influent fluid, and effluent fluid into and/or out of the growth chamber to facilitate biomass growth. In some embodiments, the first and second semi-permeable surfaces may be transparent or translucent to visible light. In some embodiments, the adsorbent material layer may become translucent to visible light responsive to being wetted by the influent fluid. In some embodiments, the adsorbent material layer may comprise a fibrous material including one or more of tissue paper, cotton, or a sponge. In some embodiments, the adsorbent material layer may be between about 30 μm and about 1 cm thick. In some embodiments, the first and second semi-permeable surfaces may comprise PDMS. In some embodiments, the first and second semi-permeable surfaces may be between about 20 μm and about 1 cm thick. In some embodiments, operation 304 may be performed by first and second semi-permeable surfaces that are the same as or similar to first and second semi-permeable surfaces 18 and 22 (shown in FIG. 1 and described herein).

For applications relating to algal cultivation, for example, the use of PDMS material, and PDMS material of this particular thickness, for a semi-permeable membrane are advantageous given the intended operating regime for growing activity. For example, during periods of peak sunlight, there approximately 10̂17 photons /(cm̂2 sec) may be received on a given (e.g., flat and/or planar) surface. This intensity of sunlight may be too high for efficient photosynthetic activity. However, in the present system, the chambers 12 may be oriented vertically to decrease this flux intensity to one-tenth of its peak to approximately 10̂16/(cm̂2*sec). At this intensity, only about 40% of the photons are in photosynthetic active wavelengths and photosynthetic activity is limited to only about 25% at this peak efficiency, leading to 10̂15/(cm̂2*sec) of photons used in the photosynthetic process. Stoichiometrically, about eight photons are required to consume one molecule of carbon dioxide and release one molecule of oxygen. So at peak efficiency, we can expect 1.25*10̂14 cm̂sec of CO₂ molecules that need to permeate the membrane and a similar amount of oxygen molecules that need to diffuse out of the device from the membrane.

Experiments performed using components similar to and/or the same as those described herein showed permeation of about 1-2 mg of water through a test device comprising a 300 cm̂2 membrane. This means that roughly 2*10̂17 /cm̂2*sec molecules of water can diffuse through the membrane. Given that the relative permeability of carbon dioxide and oxygen are about one hundredth of the permeability of water, this equates to roughly 2*10̂15 molecules/(Cm̂2*sec) of carbon dioxide and oxygen that could diffuse through. All this suggests that system 10 (e.g., using the PDMS and/or other materials in the thicknesses and arrangement described herein) is not limited by mass transfer either through being starved of carbon dioxide and/or being over-saturated with oxygen (given permeability is inversely related to membrane thickness). Additionally, it is well-known that there are very few other materials as permeable as PDMS for water, carbon dioxide, and/or oxygen, and in particular ones that might fulfill mass transfer requirements for operations similar to and/or the same as those described herein at operable thicknesses for membrane materials.

At an operation 306, the influent fluid may be provided to the growth chamber. In some embodiments, the influent device may provide the influent fluid to the growth chamber via a first needle that couples the influent device to the growth chamber. In some embodiments, the influent device may be configured to provide a continuous flow of influent fluid. In some embodiments, the influent device comprises a flow controller configured to control a flow rate of the influent fluid to the growth chamber. In some embodiments, operation 306 may be performed by an influent device the same as or similar to influent device 26 (shown in FIG. 1 and described herein).

At an operation 308, the effluent fluid from the growth chamber may be received. In some embodiments, the effluent device may receive the effluent fluid from the growth chamber via a second needle that couples the growth chamber to the effluent device. In some embodiments, the effluent device is configured to collect the biomass from the effluent fluid. In some embodiments, operation 1008 may be performed by an effluent device that is the same as or similar to effluent device 28 (shown in FIG. 1 and described herein).

At an operation 310, the growth chamber, the influent device, and the effluent device may be supported with the support structure. The growth chamber, the influent device, and the effluent device may be supported such that the influent fluid passively flows from the influent device into the growth chamber and the effluent fluid passively flows from the growth chamber into the effluent device; gases passively move through the first and second semi-permeable surfaces into and out of the growth chamber; and visible light passes through the first and second semi-permeable surfaces into the growth chamber. In some embodiments, the support structure may be configured such that the passive influent fluid flow from the influent device into the growth chamber and the passive effluent fluid flow from the growth chamber into the effluent device are caused by gravity. In some embodiments, the support structure may be configured such that the growth chamber is oriented vertically, the influent device is positioned above the growth chamber, and the effluent device is positioned below the growth chamber. In some embodiments, operation 310 may be performed by a support structure that is the same as or similar to support structure 30 (shown in FIG. 1 and described herein).

Although the disclosure has been described in detail for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A photobioreactor system comprising:
a growth chamber configured to grow a biomass, the growth chamber comprising a layered structure, the layered structure including an adsorbent material layer coupled to a first semi-permeable surface on a first side of the adsorbent material layer and a second semi-permeable surface on a second side of the adsorbent material layer, the second side opposite the first side,
wherein the first and second semi-permeable surfaces facilitate biomass growth in the adsorbent material layer by permitting passive input and output of fluid and gases to and from the growth chamber;
an influent device configured to provide influent fluid to the growth chamber;
an effluent device configured to receive effluent fluid from the growth chamber; and
a support structure configured to support the growth chamber, the influent device, and the effluent device such that:
the influent fluid passively flows from the influent device into the growth chamber and the effluent fluid passively flows from the growth chamber into the effluent device;
gases passively move through the first and second semi-permeable surfaces into and out of the growth chamber; and
visible light passes through the first and second semi-permeable surfaces into the growth chamber.

2. The photobioreactor system of claim 1, wherein the support structure is configured such that the passive influent fluid flow from the influent device into the growth chamber and the passive effluent fluid flow from the growth chamber into the effluent device are caused by gravity.

3. The photobioreactor of claim 1, wherein the support structure is configured such that the growth chamber is oriented vertically, the influent device is positioned above the growth chamber, and the effluent device is positioned below the growth chamber.

4. The photobioreactor of claim 1, wherein the biomass is algae.

5. The photobioreactor of claim 1, wherein the first and second semi-permeable surfaces permit passive input and output of carbon dioxide, oxygen, water vapor, the influent fluid, and the effluent fluid into and/or out of the growth chamber to facilitate biomass growth.

6. The photobioreactor of claim 1, wherein the first and second semi-permeable surfaces are transparent or translucent to visible light.

7. The photobioreactor of claim 1, wherein the adsorbent material layer becomes translucent to visible light responsive to being wetted by the influent fluid.

8. The photobioreactor of claim 1, wherein the adsorbent material layer comprises a fibrous material including one or more of tissue paper, cotton, or a sponge, and wherein the adsorbent material layer is between about 30 μm and about 1 cm thick.

9. The photobioreactor of claim 1, wherein the first and second semi-permeable surfaces comprise PDMS and are between about 20 μm and about 1 cm thick.

10. The photobioreactor of claim 1, wherein the influent device provides the influent fluid to the growth chamber via a first needle that couples the influent device to the growth chamber, and wherein the effluent device receive the effluent fluid from the growth chamber via a second needle that couples the growth chamber to the effluent device.

11. The photobioreactor of claim 1, wherein the influent device is configured to provide a continuous flow of influent fluid.

12. The photobioreactor of claim 1, wherein the influent device comprises a flow controller configured to control a flow rate of the influent fluid to the growth chamber.

13. The photobioreactor of claim 1, wherein the effluent device is configured to collect the biomass from the effluent fluid.

14. A method for growing a biomass with a photobioreactor system, the photobioreactor system comprising a growth chamber, an influent device, an effluent device, and a support structure, the method comprising:
forming, with the growth chamber, a layered structure, the layered structure including an adsorbent material layer coupled to a first semi-permeable surface on a first side of the adsorbent material layer and a second semi-permeable surface on a second side of the adsorbent material layer, the second side opposite the first side,
facilitating, with the first and second semi-permeable surfaces, biomass growth in the adsorbent material layer by permitting passive input and output of fluid and gases to and from the growth chamber;
providing, with the influent device, influent fluid to the growth chamber;
receiving, with the effluent device, effluent fluid from the growth chamber; and
supporting, with the support structure, the growth chamber, the influent device, and the effluent device such that:
the influent fluid passively flows from the influent device into the growth chamber and the effluent fluid passively flows from the growth chamber into the effluent device;
gases passively move through the first and second semi-permeable surfaces into and out of the growth chamber; and
visible light passes through the first and second semi-permeable surfaces into the growth chamber.

15. The method of claim 14, wherein the passive influent fluid flow from the influent device into the growth chamber and the passive effluent fluid flow from the growth chamber into the effluent device are caused by gravity.

16. The method of claim 14, further comprising orienting the growth chamber vertically, positioning the influent device above the growth chamber, and positioning the effluent device below the growth chamber.

17. The method of claim 14, further comprising permitting passive input and output of carbon dioxide, oxygen, water vapor, the influent fluid, and the effluent fluid into and/or out of the growth chamber to facilitate biomass growth.

18. The method of claim 14, wherein the first and second semi-permeable surfaces are transparent or translucent to visible light.

19. The method of claim 14, wherein the adsorbent material layer becomes translucent to visible light responsive to being wetted by the influent fluid.

20. The method of claim 14, wherein the adsorbent material layer comprises a fibrous material including one or more of tissue paper, cotton, or a sponge, and wherein the adsorbent material layer is between about 30 μm and about 1 cm thick.

21. The method of claim 14, wherein the first and second semi-permeable surfaces comprise PDMS and are between about 20 μm and about 1 cm thick.

22. The method of claim 14, further comprising providing the influent fluid to the growth chamber via a first needle that couples the influent device to the growth chamber, and receiving the effluent fluid from the growth chamber via a second needle that couples the growth chamber to the effluent device.

23. The method of claim 14, further comprising providing a continuous flow of influent fluid.

24. The method of claim 14, further comprising controlling a flow rate of the influent fluid to the growth chamber.

25. The method of claim 14, further comprising collecting, with the effluent device, the biomass from the effluent fluid.
